(19) United States
(12) Patent Application Publication
(10) Pub. No.: US 2002/0179089 A1
(43) Pub. Date: Dec. 5, 2002

(54) Portable gas heating apparatus for attachment to a pressurized gas source and method thereof

(76) Inventors: William B. Morgan, Santa Barbara, CA (US); Michael F. Ward, Panama City, FL (US); Trent M. Schultz, Goleta, CA (US); Peter M. Ryan, Orcutt, CA (US)

Correspondence Address: CISLO & THOMAS, LLP, 233 WILSHIRE BLVD, SUITE 900, SANTA MONICA, CA, 90401-1211, US

(21) Appl. No.: 09/783,928

(22) Filed: Feb. 14, 2001

(51) Int. Cl. A62B 9/00; F16K 31/26; A62B 7/04; A61M 16/00; A62B 7/00; F24J 3/00

(52) U.S. Cl. 128/204.26; 128/205.22; 128/204.17

(57) ABSTRACT

A method and system for breathing gas heat exchange for use with an underwater pressurized gas source connected to a breathing apparatus comprising a length of tubing sufficient in diameter and length to allow heat exchange of expanding gas from the pressurized source to the breathing apparatus in order to substantially increase the temperature of the expanding gas. The tubing being made of a heat conducting material and being wholly unenclosed and open for contact with ambient fluid within which the method and system is used.

### BACKGROUND OF THE INVENTION

1. Field of the Invention

The present invention relates generally to a self-contained, portable breathing gas warming system to be used by a scuba diver and more particularly to a method of warming a compressed breathing gas from a pressurized gas source using a heat exchanger immersed in the ambient seawater.

2. Description of the Related Art

The refrigeration effect of gas pressure reduction in open circuit SCUBA is little understood and has not been the subject of published information due to the lack of investigation on the subject. The refrigeration effect has been recognized as a factor of mechanical failure of the demand regulator when SCUBA diving in cold water, but the exact cause has not been defined. While prior art designs to overcome this problem have focused on the second stage demand regulator, we have found that the biggest part of the problem is at the first stage pressure reduction regulator. SCUBA diving regulators are well known in the art. Typically, they constitute one or more tanks of compressed air(or mixed gas) with two stages of air pressure regulation/reduction. The first stage regulator reduces high pressure air of 2,000 to 6,000 psi to about 150 psi low pressure air. The second stage regulator provides low pressure air to the diver's respiratory system at ambient pressure, upon demand, to enable the diver to breathe normally under water. For each breathing cycle, high-pressure air flows through the first stage regulator valve orifice and is reduced from 2,000 psi (or higher) to 150 psi. As this gas flows through and around the valve mechanism of the first stage regulator it rapidly expands and flows through the low-pressure hose to the second stage demand regulator. This rapid pressure drop and expanse of gas at the first stage causes a cooling condition. If SCUBA diving in water at 75° F., for example, the first stage valve mechanism and housing of the regulator can become cooled below the freezing point of water. The relative warm (75° F. for example) surrounding water prevents freeze up of the regulators, but the diver's mouth, throat and lungs must deal with heating the cold air of each breath. This cools the diver rapidly although there is little or no awareness to the diver that the inspired air is cold.

When diving in cold water (below 40° F. for example) the extreme cold air flowing from the regulator becomes a serious problem. Temperatures well below freezing can be flowing to the diver from the second stage regulator. At these temperatures there is an immediate danger to the diver in two ways. First, the regulator may mechanically freeze up quickly, threatening the diver's supply of breathing gas. Second, the cold air hitting the diver's mouth, throat, and airways presents a real danger of causing respiratory shock, which results in the diver not being able to breathe.

Prior art SCUBA first stage regulators utilize captured freeze resistant fluids in their mechanisms to avoid freeze up. From the first stage, the super cooled air travels through a hose to the second stage demand regulator where a further reduction in air pressure causes additional cooling to the already cold air. The slightest moisture in the second stage regulator housing, either from exhaled breath or the surrounding environment will condense and freeze on these super cooled parts causing an icing condition within the regulator housing. Ice can continue to build up to the point where it can block the mechanism from proper operation. The valve mechanism freezes in an open position bringing about even more cooling and freezing and thereby causing a dangerous breathing condition in addition to a rapid depletion of the diver's gas supply. As a result there has been a need for an improved breathing system that will reduce these problems.

Heat exchangers have been used heretofore to permit warming of the breathable pressurized air as for example, in Marcus, U.S. Pat. No. 3,898,978, Aug. 12, 1975, which discloses a heat exchanger comprising a vortex tube used for supplementary heating of the compressed breathing gas, which requires many specific structures and complications to its effectiveness and economic use.

As in the afore cited Marcus Patent, Marcus U.S. Pat. No. 4,014,384, Mar. 29, 1977, also discloses a heat exchanger to heat the compressed breathing gas with the elimination of the vortex tube. While their patent discloses a small tubular extension of the gas flow exposed to the ambient water, it also requires an enclosing container or chamber. Ordinarily, the hose, which is usually rubber or insulated is not effective as a heat exchange. Both patents, however, disclose the heat exchanger inside an insulated container containing a preheated fluid through which the breathing gas circulates. This fluid is added just before the dive or a self-contained unit can be added to the system to heat the fluid. In either event it requires support equipment to make the fluid hot. This is an inconvenience and adds additional cost and time because one must supply a heating source and wait until the fluid is heated before commencing the dive. Still further, the Marcus Patents disclose the heat exchanger encased in an insulated container that does not allow for breathing gas temperature exchange between the exchanger tubing and the ambient liquid that the diver is immersed in.

Another problem with the prior art is that the need for a preheated liquid limits the time for the dive because the heating process can only be maintained for a period between one and three hours. The present invention has no limit since there is no need for a preheated liquid.

Therefore, it would be highly desirable to provide a system using a heat exchanger which does not require any support equipment, is ready at any time, does not limit the diving time and allows usage of the ambient liquid which the diver is immersed in to heat the compressed breathing gas. In other words, a system which is simpler and more economical.

The present invention provides a system which achieves these goals. In brief summary, the present invention allows for a compressed gas to be warmed near the temperature of the surrounding ambient or immersed liquid without use of support systems, does not limit dive time and can be ready for use at any time.

### SUMMARY OF THE INVENTION

A system using a heat exchanger which does not require any heating support equipment, is ready at any time and allows usage of the ambient liquid, namely seawater, in which the diver is immersed in to heat the compressed breathing gas is described.

In order to accomplish this goal, what is needed is an exposed heat exchanger system allowing the liquid that the diver is immersed in to be the warming factor in the heat exchanging process.

The heat exchanger of the invention is designed to heat the cooled gas close to the temperature of the surrounding water in which the diver is immersed. This provides, without supplemental heating or power, a temperature gain from any cooled temperature to ambient water temperature. The temperature of the tank of compressed breathing gas the diver carries is the same as the surrounding or ambient water. The compressed gas is cooled to temperatures below freezing when expanding through the first stage pressure reduction regulator. Then the low-pressure gas that has been cooled flows through a heat exchanger system, secured to the air tanks on the diver, and the breathing gas is warmed to around ambient water temperature.

The breathing gas, now close to ambient water temperature, flows to the demand regulator where a slight reduction in pressure, from 150 psi over ambient to ambient takes place. This small reduction in pressure(when compared to reducing from tank pressures of 2000 psi or more to 150 psi) does cool the breathing gas, but only slightly, well within the range of the mechanical device and human functions.

In particular, one embodiment of the invention is directed to an unenclosed, open system where the metal tubing of the heat exchanger is comprised of tubing which is about ½″ in diameter and 6′ long. Yet other embodiments of the present invention have the tubing diameter ranging from ¼″ to 3″ and from 3″ to 10′ in length. In yet other embodiments the metal tubing of the heat exchanger is comprised of copper, stainless steel or brass.

An object of the invention is to provide a method and apparatus to allow a diver to breathe compressed gas at a temperature as close to the immersed liquid as possible without the need for support systems or a heated liquid encompassed within the system. Another object of the present invention is not to limit the time of the dive because of support systems or heated liquids that are necessary in the system.

It is still another object of the invention to provide an underwater breathing system using compressed gas where the compressed gas is reduced in pressure and made to flow through a heat exchange system wholly disposed in the ambient water in which the system is used.

It is still another object of the invention to provide an apparatus for use in underwater heating systems where a heat exchanger component carrying exposed tubing of sufficient size and length is connected within the system and exposed to the free flowing currents of ambient water with which the underwater breathing system is utilized to thereby allow gas used in the system to be warmed relative to the ambient water.

These and other objects and advantages of the present invention will become apparent from a reference to the following specification and accompanying drawings wherein the numerals of reference designate like elements throughout.

### BRIEF DESCRIPTION OF THE DRAWINGS

FIG. 1 is a perspective view showing the diver wearing SCUBA gear incorporating the heat exchanger component of the present invention.

FIG. 2 is a partial perspective view of the heat exchanger component of the present invention excluding the cover assembly, shown in association with typical or conventional SCUBA equipment;

FIG. 3 is an exploded, perspective view of the main components of the heat exchanger of the invention showing the manifold, cover assembly and metal tubing;

FIG. 4A is a front view of the metal tubing making up the heat exchange component of the invention;

FIG. 4B is a side view of the metal tubing depicted in FIG. 4A; and

FIG. 4C is a top view of the metal tubing shown in FIG. 4A.

### DESCRIPTION OF THE PREFERRED EMBODIMENT(S)

The detailed description set forth below in connection with the appended drawings is intended as a description of presently preferred embodiments of the invention and is not intended to represent the only forms in which the present invention may be constructed and/or utilized. The description sets forth the functions and the sequence of steps for constructing and operating the invention in connection with the illustrated embodiments. However, it is to be understood that the same or equivalent functions and sequences may be accomplished by different embodiments that are also intended to be encompassed within the spirit and scope of the disclosed invention.

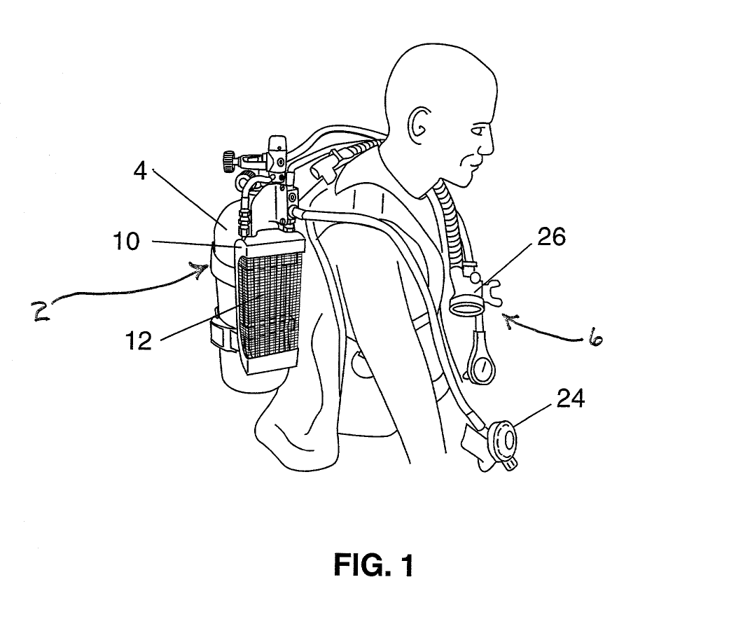

FIG. 1, shows a gas exchange device 2 for use with an underwater pressurized gas source 4 connected to a breathing apparatus 24 and/or 6. The gas exchange device 2 comprises a combination of a length of tubing 8 sufficient in diameter and length to allow heat exchange of expanding gas from the pressurized gas source 4 to the breathing apparatus 24 and/or 6 in order to substantially increase the temperature of the breathing gas. The tubing 8 is made of a heat conducting material and is wholly unenclosed and open for contact with ambient fluid within which the gas exchange device 2 is used. Ideally, to obtain maximum surface area exposure in as little space as possible, the tubing is configured in a serpentine shape. In the preferred embodiment, the tubing 8 is protected by a cover assembly 10 with an open grating 12 which allows contact between the tubing 8 and the ambient fluid. However, if preferred, the tubing 8 not need be protected by grating 12. In the preferred embodiment, the heat conducting material of tubing 8 is copper, but in other embodiments it may be made of stainless steel or other suitable heat conducting materials.

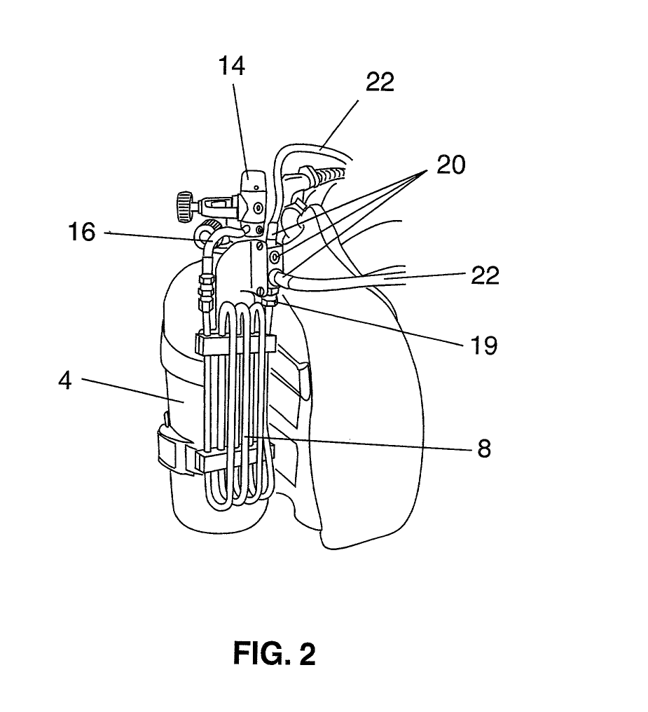

In FIG. 2, the present invention in conjunction with all of the SCUBA equipment is shown with the exception of the cover assembly. The breathing gas emanates from the pressurized gas source 4 and flows to the first stage regulator 14, which is a gas pressure reduction regulator. This first stage regulator 14 reduces the pressure of the breathing gas from 2,000 psi or above to approximately 150 psi, which in turn causes a significant decrease in temperature of the breathing gas. It then passes through an inlet tube 16, which in the preferred embodiment is six inches in length, which connects to the tubing 8, which is surrounded by the ambient fluid that the diver is immersed in, typically seawater although it may be fresh water. As the compressed breathing gas passes through the tubing 8, the temperature rises to near the temperature of the ambient fluid that the diver is immersed in. No supplemental power is needed in the present invention, the compressed breathing gas is heated by flowing through the tubing 8 which is in contact with the ambient water in which the diver is immersed. In the preferred embodiment, at the exit of the tubing 8, it travels through a manifold block 18, which is attached to the tubing 8. This manifold block 18 is comprised of an inlet port 19 and has multiple outlet ports 20 allowing for the warmed, compressed breathing gas to flow from the tubing 8 and through outlet tubes 22 to a second stage regulator 24, from which the diver inhales, and a backup second stage regulator and/or power inflator 26. However, other means can be used to transport the compressed breathing gas from the tubing 8 to the second stage regulator.

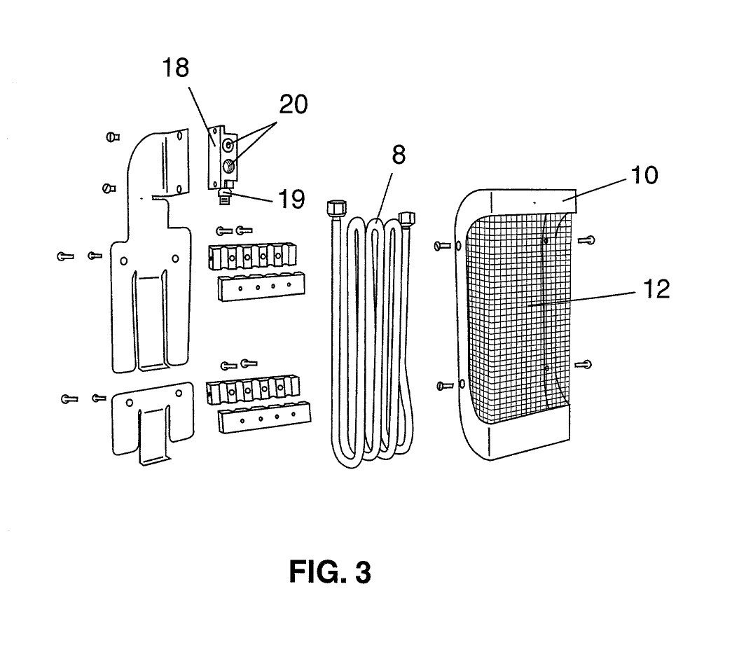

FIG. 3 shows the manifold block 18, tubing 8, and the cover assembly 10 of the present invention. The cover assembly 10 has an open grating 12 which allows the surrounding water(in which the diver is swimming) to freely flow over and around the tubing 8 to warm the compressed breathing gas that has been cooled by expanding out of the first stage regulator 14.

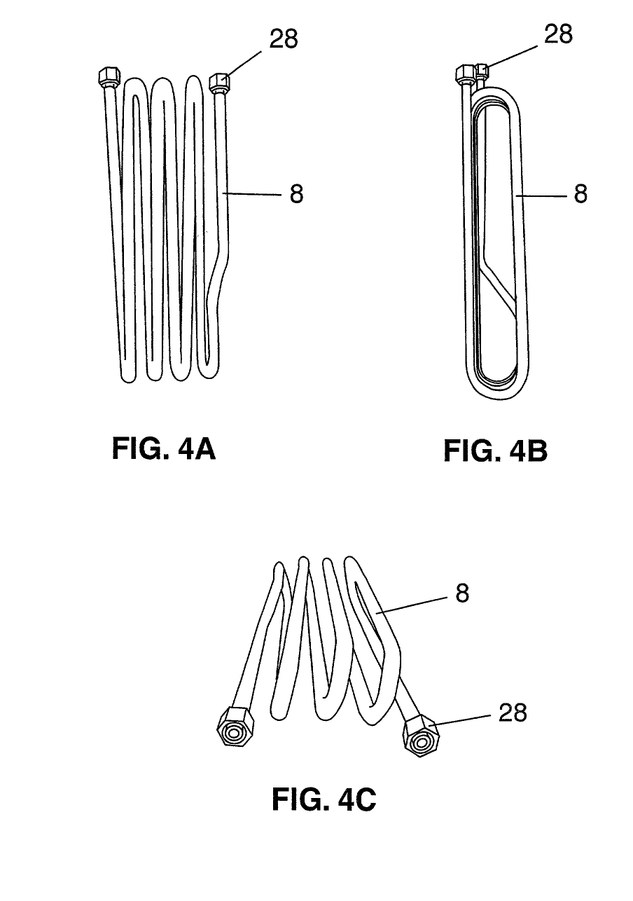

FIGS. 4A, 4B, and 4C, show the tubing 8, which is the tubing 8 of the gas exchange device of the present invention. In the preferred embodiment, the tubing 8 consists of copper. The tubing 8 can also consist of other heat conducting materials including stainless steel. In the preferred embodiment, the tubing 8 is about ½″ in diameter and about 6′ in length. However, the diameter of the tubing 8 can range from about ¼″ to 3″ and the length can range from about 3″ to 10′. Each end of the tubing 8 contains an end fitting 28 which allows for attachment of parts that facilitate flow of the breathing gas.

Tests were performed which show the improved results that one obtains with the present invention. Each test was performed in cold water using an aluminum SCUBA bottle (filled with standard breathing air), a standard first stage regulator with a SCUBA hose connected in LP port to the tubing 8, all connected to a second stage regulator cycling machine that was out of the water. The tests being labeled 1 through 5. Test 1 is performed without the use of the present invention. Instead, a standard 28″ SCUBA hose is used. Tests 2 through 5 are conducted with hose 16, being 6″ long and various lengths of tubing 8. Tubing 8 was composed of ⅜″ diameter copper ranging from 2′ to 8′ in length. The results show that the present invention causes significant rise in the temperature of the compressed breathing gas so as not to result in mechanical equipment failure and/or harm to the diver. As the length of the tubing 8 increases from 2′ to 8′ the temperature at the outlet side of the tubing 8 more closely approaches the ambient fluid temperature. The results were tabulated with an overview of the entire results at the end. The results are as follows: (all temperatures are in Fahrenheit)

Test 1: SCUBA Hose Only
Water Temp: 40.8°

| PSI | 1^{st} Stage | Out end of Hose |
|---|---|---|
| 2000 | 25.0° | 16.5° |

Note: At the end of the test the first stage had a ½″ layer of ice on the outside of the LP out side of the regulator (not the piston), and the second stage (metal) that is mounted on the breathing machine that is not in the water was so cold it had frost all over the inlet side of the regulator.

Test 2: 2′ Heat Exchanger
Water Temp: 40.1°

| PSI | 1^{st} Stage | Heat Exchanger In | Heat Exchanger Out |
|---|---|---|---|
| 2000 | 32.9° | 11.2° | 31.8° |

Note: At the end of the test the first stage had a ½″ layer of ice on the outside of the LP out side of the regulator (not the piston), the heat exchanger had ¼″ of ice that tapered down to the tube for about 10″, second stage (metal) that is mounted on the breathing machine that is not in the water was not as cold at the first test and did not freeze the moisture that was on the outside of the inlet side of the regulator.

Test 3: 4′ Heat Exchanger
Water Temp: 39.6°

| PSI | 1^{st} Stage | Heat Exchanger In | Heat Exchanger Out |
|---|---|---|---|
| 2000 | 34.9° | 11.1° | 35.8° |

Note: At the end of the test the first stage had a ½″ layer of ice on the outside of the LP out side of the regulator (not the piston), the heat exchanger had no ice on it at all this time, second stage (metal) that is mounted on the breathing machine that is not in the water was not as cold at the first test and did not freeze the moisture that was on the outside of the inlet side of the regulator.

| Test 4: 6′ Heat Exchanger | | | |
|---|---|---|---|
| Water Temp: 39.2° | | | |
| PSI | 1^{st} Stage | Heat Exchanger In | Heat Exchanger Out |
| 2000 | 34.0° | 9.9° | 37.6° |

Note: At the end of the test the first stage had a ½″ layer of ice on the outside of the LP out side of the regulator (not the piston), the heat exchanger had no ice on it at all this time, second stage (metal) that is mounted on the breathing machine that is not in the water was not as cold as the first test and did not freeze the moisture that was on the outside of the inlet side of the regulator.

| Test 5: 8′ Heat Exchanger | | | |
|---|---|---|---|
| Water Temp: 38.8° | | | |
| PSI | 1^{st} Stage | Heat Exchanger In | Heat Exchanger Out |
| 2000 | 34.9° | 11.0° | 38.6° |

Note: At the end of the test the first stage had a ½″ layer of ice on the outside of the LP out side of the regulator (not the piston), the heat exchanger had no ice on it at all this time, second stage (metal) that is mounted on the breathing machine that is not in the water was not as cold at the first test and did not freeze the moisture that was on.

Over View

Water Temp: 39.5° 39.5° 40.1° 39.6° 39.2° 38.8°

| PSI | Ist Stage | In | 2′Out | 4′Out | 6′Out | 8′Out |
|---|---|---|---|---|---|---|
| 2000 | 32.0° | 10.0° | 31.8° | 35.8° | 37.6° | 38.6° |

Note: The temperatures listed in the 1^{st} Stage column and the “In” column are averages.

While the present invention has been described with regards to particular embodiments, it is recognized that additional variations of the present invention may be devised without departing from the inventive concept.

What is claimed is:

1. A gas heat exchange device for use with an underwater pressurized gas source connected to a breathing apparatus comprising the combination of:
a length of tubing sufficient in diameter and length to allow heat exchange of cooled gas from said pressurized source to said breathing apparatus to substantially increase the temperature of said breathing gas, said length of said tubing being made of a heat conducting material and being wholly exposed and open for contact with ambient fluid within which said device is used.

2. The apparatus as set forth in claim 1, wherein said ambient fluid is water.

3. The apparatus as set forth in claim 2, wherein said expanding gas flows from said pressurized gas source to said breathing apparatus step wise through:
a) a first stage regulator,
b) said inlet tube,
c) said length of said tubing,
d) a manifold block with multiple outlet ports,
e) a plurality of outlet tubes, and
f) a second stage regulator.

4. The apparatus as set forth in claim 1, wherein a protective housing with an open grating connectively attaches to said multiple out ports and covers said tubing.

5. The apparatus as set forth in claim 4, wherein said tubing is about ½″ in diameter, is about 6′ in length and is made of copper.

6. The apparatus as set forth in claim 4, wherein said tubing contains end fittings allowing said inlet tube and said multiple out ports to be attached thereto.

7. The method of increasing the temperature of an expanding gas from a pressurized tank used in a SCUBA system comprising the steps of:
allowing said gas from said pressurized tank to expand and to flow through a length of tubing sufficient in diameter and length to allow heat exchange of expanding gas from said pressurized tank to substantially increase the temperature of said gas, said length of said tubing being made of a heat conducting material and being wholly unenclosed and open for contact with ambient fluid within which said method is used.

8. The method as set forth in claim 7, wherein said ambient fluid is water.

9. The method as set forth in claim 8, wherein said tubing is about ½″ in diameter, is about 6′ in length and is made of copper.

10. A gas heat exchange device for use in scuba apparatus using an underwater pressurized gas source connected to a breathing apparatus comprising the combination of:
a length of metal tubing configured in serpentine shape and operatively connected to said pressurized gas source and being sufficient in diameter and length to allow heat exchange of cooled gas from said pressurized source to said breathing apparatus to substantially increase the temperature of said breathing gas, said length of said tubing being made of a heat conducting material and being wholly exposed and open for contact with ambient fluid within which said device is used, and
a protective open, grid-like member encompassing at least a portion of said tubing in protective relationship therewith.
